(19) United States
(12) Patent Application Publication
(10) Pub. No.: US 2005/0118720 A1
(43) Pub. Date: Jun. 2, 2005

(54) Method for the detection of proteins of animal origin in complex mixtures

(76) Inventors: Carlos Bloch Jr, Brasilia (BR); Clarissa Silva Pires De Castro, Brasilia (BR); Maura Vianna Prates, Brasilia (BR)

Correspondence Address: SUGHRUE MION, PLLC, 2100 PENNSYLVANIA AVENUE, N.W., SUITE 800, WASHINGTON, DC, 20037, US

(21) Appl. No.: 10/504,590

(22) PCT Filed: Jul. 3, 2002
(86) PCT No.: PCT/BR02/00092

(30) Foreign Application Priority Data
Mar. 4, 2002 (BR) PI 0200690-1

(51) Int. Cl. G01N 33/02

(52) U.S. Cl. 436/20

(57) ABSTRACT

The purpose of the present invention is to evaluate the quality of feed for ruminants and consequently, avoid the transmission of TSEs through the detection of animal proteins in these foods. This purpose is embodied in the form of a method for detecting proteins of animal origin in complex mixtures comprising the stages of: (i) extraction of the proteic matter in high concentration from a sample of the initial complex mixture in a manner as to substantially remove all interferents; (ii) preparation of the matrix-analyte in a manner as to maintain low levels of impurities and an adequate matrix-analyte molar rate; (iii) analysis of the material obtained in the prior stage by MALDI-TOF mass spectrometry; (iv) optionally, fractionation of the samples or isolation of the components by RP-HPLC and identification of the components by means of automatic sequencing of the N-terminal region and sequencing of its peptidic fragments by liquid chromatography coupled to mass spectrometry (LC/MS/MS). The present invention also contemplates the use of this method in the detection of proteins of animal origin in feed for ruminants, which permits the interruption of transmission of Transmittable Spongiform Encephalopathies, and more particularly Bovine Spongiform Encephalopathies (BSE).

### FIELD OF THE INVENTION

The present invention refers to a method for the detection of proteins of animal origin in feed intended for mammals, specially for ruminants, with the aim of monitoring the quality of the food and avoiding the transmission of diseases caused by infectious substances, such as Prions that transmit Transmittable Spongiform Encephalopathies (TSE) and more specifically, Bovine Spongiform Encephalopathies (BSE), known as the “Mad Cow Disease”.

### BACKGROUND OF THE INVENTION

Transmittable Spongiform Encephalopathies (TSE) are progressive and lethal diseases that affect the central nervous system and are characterised by anatomical alterations localised in the brain. These alterations are a type of histologic lesion constituted of proteic deposits and vacuoles. TSE may result from spontaneous infection, hereditary transmission or, for example, iatrogenic exposure to contaminated material. Some TSEs include:
Bovine Spongiform Encephalopathies (BSE), or “Mad Cow Disease”.
Scrapie, or ovine enzootic paraplegia, which affects ovines and caprines in many countries and has been known for more than 200 years.
Creutzfeldt-Jacob Disease (CJD) which affects human, normally ageing patients, is distributed world wide, with an annual incidence of approximately one case per million and occurs in three forms:
   Sporadic—responsible for 85-90% of the cases;
   Familiar—associated to genetic mutations, representing 5-10% of the cases;
   Iatrogenic—responsible for less than 5% of the cases.
New variant of the CJD (vCJD), which, in contrast to the traditional forms of CJD, affects young patients. There is strong evidence that the vCJD results from consuming bovine products infected by BSE.

The nature of the infectious agent of BSE, as well as the other TSEs, remains the cause of controversy. It is believed that the infectious particles responsible for TSE are predominantly specific proteins called Prions, which are composed, almost entirely, by a glycoprotein of abnormal conformation, which attaches itself to the external surface of the cells (U.S. Pat. No. 6,197,207). In other words, the most accepted theory postulates that the Prion infectious agent would have derived from a protein from the cellular membrane sensitive to the protease (PrPc), which would suffer a change in conformation to form an insoluble and pathogenic type of Prion (PrPsc). In turn, the PrPsc protein would induce the transformation of more normal proteins to becoming abnormal forms, starting a chain reaction that would increase the production of PrPsc in an exponential manner. The mechanism by which the abnormal proteins produce the pathological alterations in the brain of the affected individuals or animals is not entirely clear. This theory has a weakness; various forms of Scrapie are known, characterised by having different incubation periods, clinical signs and pathologies, which is more consistent with the theory that these infirmities are caused by an infectious agent of the viral type.

The first cases of BSE were diagnosed in the United Kingdom in 1986 and by late 1987 the Department of Epidemiology of the Central Veterinary Laboratory concluded that the dissemination of the disease in the bovine population occurred through the consumption of meat and bone meal, obtained from the carcasses of contaminated animals and incorporated to the feed of the bovines. This theory was fully confirmed when a clear effect was noted after prohibiting the use of this product for the feeding of ruminants, which resulted in a sharp decline of the number of new cases of BSE. Other forms of dissemination have not been fully demonstrated, but cannot be discarded, such as, for example, the vertical transmission from the cow to the calf. However, it is known that the BSE epidemic would not have occurred if there had not been dissemination through meat and bone meal. Thus, if a contaminated bovine is introduced to a country—or region—where there is no BSE, an epidemic can only occur if the carcass of that animal is used to make meal intended for the feeding of ruminants and therefore generating a dissemination and amplification system for the infectious agent in the animal population.

After the beginning of the epidemic in the United Kingdom, a theory arose that the first cases of BSE were caused by the use of ovine carcasses contaminated with Scrapie in the feeding of bovines and that an alteration in the industrial process for producing meat and bone meal would have reduced the probability of deactivating the infectious agent. However, evidence began to appear that BSE and Scrapie are distinct diseases, despite belonging to the same group of infirmities, because: (i) Scrapie when inoculated experimentally in bovines produces a different disease to BSE; (ii) BSE maintained its characteristics throughout the whole epidemic, even when crossing the barrier of species (transmission to other animal species), which does not occur with Scrapie; (iii) there is no epidemiological evidence to this day that Scrapie can contaminate human beings.

The most recent technical-scientific reports indicate that the cases of BSE diagnosed since 1986 were not the first cases of the disease, which probably already existed in the United Kingdom beforehand. Some British veterinarians claim to have seen similar cases before 1986, which at the time were diagnosed as metabolic diseases common in high production cows. The theory that the change in the industrial production process of meat and bone meal was responsible for recycling the infectious agent is also being questioned since there is increasing evidence that none of the processes employed would be capable of deactivating the agents causing TSE. In fact, the TSE infections have the uncommon property of a high resistance to physical-chemical sterilisation treatments (U.S. Pat. No. 6,197,207).

Food based on bone and meat have been widely recommended and employed in the feeding of animals as a source of protein due to the presence of essential amino acids, minerals and vitamin B12. Furthermore, such use is an efficient manner to utilise the waste resulting from slaughter, which thus avoids additional economical and environmental costs. However, as materials based on meat and bone from mammalians present in the feed for ruminants was considered the probable cause of BSE in bovines, their use in the feeding of ruminants was prohibited within the European Community (norms 94/449/EC; 99/129/EC) and in the USA.

There have been various manners envisaged to cease the transmission of TSEs, and particularly of BSE. Some works have been directed at the inactivation of the infectious particles, favouring thus the use of abattoir residues. Others aimed at—quite simply—eliminating the possibility of transmission by the detection of animal proteins in feed and their rejection in the case of the test proving positive. Both outlooks, however, make use of analytic procedures to guarantee the absence of agents causing the TSEs in the feeding of animals.

In this context, the following may be cited as being state of the art: (a) the ELISA and dot-ELISA immunoenzymatic methods, described in the works of Kingcombe and collaborators (see Kingcombe, C. I. B., Luthi, E., Schlosser, H., Howald, D., Kuhn, M. and Jemmi, T. (2001). “A PCR-based test for species specific determination of heat treatment conditions of animal meals as an effective prophylactic method for bovine spongiform encephalopathy”. Meat Science, 57, 35-41), of Macedo-Silva and collaborators (see Macedo-Silva, A., Barbosa, S. F. C., Alkmin, M. G. A., Vaz, A. J., Shimokomaki, M. and Tenuta-Filho, A. (2002). “Hamburger meat identification by dot-ELISA”. Meat Science, 56, 189-192) and in U.S. Pat. No. 5,910,446 which describes the detection of thermostable proteins present in feeds for ruminants based on the concentration of these proteins so as to increase the quantity of these in the samples and, therefore, increase the sensitivity of the immunotests; (b) the PCR tests as related by Kingcombe and collaborators and in U.S. Pat. No. 6,033,858 which describes the detection in samples of the specific spiroplasma fragment 16S rDNA which is indicative of TSE and (c) the mass spectroscopy (ESI-MS) described by Ponce-Alquicira (see Ponce-Alquicira, E. and Taylor, A. J. (2000). “Extraction and ESI-CID-MS/MS analysis of myoglobins from different meat species”. Food Chemistry, 69, 81-86).

Various patent documents may also be cited to illustrate this technology: U.S. Pat. No. 5,750,361 that mentions a test based on the contact of a compound to be tested with a first PrPsup.C, or variant PrP component, in the presence of a second peptidic component and then determining the capacity of the above compound to avoid the forming of a protein-prion complex, where the source of the first PrPsup.C complex may be of the human, mouse, hamster, bovine or ovine species; U.S. Pat. No. 5,846,533 which describes a test to determine the presence of prions (i.e., PrP.sup.Sc—scrapie isoform of the prion protein, agent causing spongiform encephalitis) in products such as drugs, foods derived from natural sources or similar by means of specific antibodies that bond the PrP.sup, Sc in situ, with the antibodies that only bond to the PrP.sup.Sc native to a particular species being preferred, for example, human, bovine, ovine, porcine etc.; U.S. Pat. No. 6,165,784 that describes an immunotest employing a monoclonal antibody that bonds specifically to an epitope conserved from prion proteins of ruminants, because monoclonal antibodies have the property of bonding to the epitope of the PrP gene of ruminants, identified as Ile-His-Phe-Gly that occurs in ovines (amino acids 142-145) and bovines (amino acids 150-153); U.S. Pat. No. 6,008,435 that describes the detection of bovine, ovine and human prions in a sample using a transgenic mouse having an exogenous PrP gene obtained from the above species; U.S. Pat. No. 6,114,693 relates the application of mass spectroscopy employing an ionic source for ionising compounds contained in solution for the analysis of solutions prepared with myoglobin and haemoglobin; U.S. Pat. No. 5,916,445 that describes the use of a chromatographic method (affinity chromatography) for recognising and pre-selecting of species, with, as example, the differentiation of the myoglobin chromatograms of two different species of mammals (horse and whale). It was verified that the chromatographic column prepared with the myoglobin of the horse does not adsorb the whale myoglobin. This indicates the existence of a high degree of specificity in view of the fact that the composition of amino acids of the two myoglobins differ in merely 20 of the 153 amino acids of this protein and that the three dimensional structure is affected only very slightly during the test.

The complexity and specificity of the bio-molecules has made it much more difficult to apply the techniques frequently used for the identification and characterisation of organic and inorganic compounds. This fact has motivated the development of increasingly efficient and sophisticated analytic techniques, with emphasis being accorded to the precision required by modern biotechnology.

In fact, there are instruments available today—such as mass spectrometers—that allow the detection, identification and characterisation of nucleotide sequences and of amino acids from one or more peptides. Some examples are the technique of desorption/ionisation of the analyte with the aid of an organic acid (matrix) through laser radiation (MALDI-TOF-MS) and the technique of ionisation by vaporisation of droplets of analyte solvated by a liquid mixture (spray) (ESI-MS). Preferentially, separation techniques, such as HPLC (High Performance Liquid Chromatography) or electrophoresis, are directly or indirectly coupled to the mass spectrometer.

The MALDI-TOF-MS technique is being much used in the analysis of macromolecules, especially peptides, proteins and nucleic acids. The possibility of investigating different classes of compounds is the result of the use of different and optimised combinations of matrixes and laser wavelengths. Various patent documents describe these applications in detail, of which: U.S. Pat. No. 6,235,478 and U.S. Pat. No. 6,277,573 which refer to the detection of DNA molecules with diagnosis purposes; U.S. Pat. No. 6,218,118 relates to a preparation of a mixture of compounds that allow the analysis of nucleotide sequences by mass spectrometry; U.S. Pat. No. 6,057,543 describes the improvement in spectrometer for the analysis of bio-molecules; U.S. Pat. No. 6,287,872 refers to support slides for the analysis of molecules with an elevated molecular weight; U.S. Pat. No. 6,265,716 deals with volatile matrixes for MALDI-TOF-MS spectrometry.

The document U.S. Pat. No. 6,278,794 describes the isolation and the computerised characterisation of proteins. In accordance with this method, the proteins are separated from a complex mixture by electrophoresis and, after isolating the bands, the sequencing is done using the MALDI-TOF-MS or ESI-MS technique. The disadvantage of this method is the necessity of various separation stages, which may compromise the sensitivity of the test when the concentration of the substance to be detected is very low.

The document U.S. Pat. No. 6,265,715 refers to a non-porous membrane employed as a sample support in MALDI-TOF-MS mass spectrometry for the analysis of peptides and proteins. The possibility of analysing whole blood samples is mentioned, despite not concluding that the method would work for biological fluids. In fact, the analysis is made from a mixture prepared with standard substances and under controlled conditions. The following are employed: myoglobin from horse hearts (16.951 Da), bovine insulin (5.733 Da) and bovine seroalbumin (66.430 Da) acquired from Sigma Chemicals (St. Louis, Mo., USA) and apotransferrin (78.030 Da) obtained from the Calbiochem company (LaJolla, Calif., USA). It is evident that the success in the detection of proteins from more complex mixtures is not predictable based on the procedures of U.S. Pat. No. 6,265,715 patent.

In brief, despite the various proposals to solve the problem of TSE transmission and the widespread application of computerised mass spectrometry methods, still remains a demand for trustworthy tests that allow the guarantee of the absence of animal protein in feed and consequently impede the contamination of animals free of this disease.

### SUMMARY OF THE INVENTION

The purpose of the present invention is to evaluate the quality of feed for ruminants and consequently, avoid the transmission of TSEs through the detection of animal proteins in these foods. This purpose is embodied in the form of a method for detecting proteins of animal origin in complex mixtures comprising the stages of: (i) extraction of the proteic matter in high concentration from a sample of the initial complex mixture in a manner as to substantially remove all interferents; (ii) preparation of the matrix-analyte in a manner as to maintain low levels of impurities and an adequate matrix-analyte molar rate; (iii) analysis of the material obtained in the prior stage by MALDI-TOF mass spectrometry; (iv) optionally, fractionation of the samples or isolation of the components by RP-HPLC and identification of the components by means of automatic sequencing of the N-terminal region and sequencing of its peptidic fragments by liquid chromatography coupled to mass spectrometry (LC/MS/MS). The present invention also contemplates the use of this method in the detection of proteins of animal origin in feed for ruminants, which permits the interruption of transmission of Transmittable Spongiform Encephalopathies, and more particularly Bovine Spongiform Encephalopathies (BSE).

### BRIEF DESCRIPTION OF THE DRAWINGS

FIG. 1: Shows the analytic procedure employed in testing the feeds in accordance with the method of the present invention.

FIG. 2: Shows the mass spectrum obtained by the analysis of the feed A5. The peak “A” corresponds to ions of vegetable proteins.

FIG. 3: Shows the mass spectrum obtained by the analysis of the feed G2. The peaks “A” correspond to ions of vegetable proteins and “B” to animal proteins.

FIG. 4: Shows the mass spectrum, in the 15-20 kDa region, obtained by the analysis of the feed G2. The peak “C” corresponds to swine myoglobin.

FIG. 5: Shows the results of the analyses of 185 samples of commercial rations. A=feeds with positive test results (9%); B=feeds with negative test results (81%); C=feeds with inconclusive test results.

FIG. 6: Illustrates the proportion of components corresponding to the peaks of the mass spectrum of the feeds analysed. A=8 kDa (4%); B=6 kDa (3%); C=7 kDa (28%); D=10 kDa (0%); E=9 kDa (31%); F=11 kDa (2%); G=13 kDa (10%); H=14 kDa (0%); I=16 kDa (6%); J=15 kDa (3%); L=5 kDa (8%); M=17 kDa (5%).

FIG. 7: Graphically represents the results of the analyses of eight groups (A to J) of feed, whose samples did not present positive test results, or non-analysable samples.

FIG. 8: Shows the distribution, by group, of the samples with positive test results: K (34%); L (12%); M (24%); N (12%); 1 (12%); G (6%); and I (12%).

FIG. 9: Graphically represents the results of the analyses of six groups of feed whose samples presented positive test results: K (25 samples); L (14 samples); M (10 samples); N (26 samples); I (30 samples); G (5 samples).

FIG. 10: Illustrates the result of the analyses of the feeds from group K: A=feeds with positive test results (24%); B=feeds with negative test results (24%); C=feeds with inconclusive test results (52%).

FIG. 11: Illustrates the result of the analyses of the feeds from group L: A=feeds with positive test results (14%); B=feeds with negative test results (86%); C=feeds with inconclusive test results (0%).

FIG. 12: Illustrates the result of the analyses of the feeds from group M: A=feeds with positive test results (40%); B=feeds with negative test results (60%); C=feeds with inconclusive test results (0%).

FIG. 13: Illustrates the result of the analyses of the feeds from group N: A=feeds with positive test results (8%); B=feeds with negative test results (65%); C=feeds with inconclusive test results (27%).

FIG. 14: Illustrates the result of the analyses of the feeds from group I: A=feeds with positive test results (7%); B=feeds with negative test results (76%); C=feeds with inconclusive test results (17%).

FIG. 15: Illustrates the result of the analyses of the feeds from group G: A=feeds with positive test results (20%); B=feeds with negative test results (80%); C=feeds with inconclusive test results (0%).

FIG. 16: Shows the distribution of feed samples with positive test results by type of myoglobin. A=swine myoglobin (34%); bovine myoglobin (24%); C=bovine myoglobin (MAAQ- - -AAEK) (polymorphic form, 24%); D=equine myoglobin (12%); E=equine myoglobin (D- - -N) (polymorphic form, 6%).

FIG. 17: Represents the ratio between the sample analysed and the type of myoglobin encountered in the composition. K8,K20,K21,N8,G2,M0=swine myoglobin; N1,K7,L2, M5=bovine myoglobin; K25,L1, M2=bovine myoglobin (MAAQ- - -AAEK); M6, M8=equine myoglobin; K22=equine myoglobin (D- - -N).

### DETAILED DESCRIPTION OF THE INVENTION

So as to facilitate the comprehension of the present invention, definitions of important terms relating to the techniques involved in the method of detection are supplied below:
Complex mixture includes products containing principally organic substances of animal and/or vegetable origin and additives frequently employed in solid or liquid animal foods.
Interferents are components that are present in the initial complex mixture and that render more difficult any conclusive analysis for the presence of proteins of animal origin by spectrometric methods, such as, for example, MALDI-TOF mass spectroscopy, and include substances such as carbohydrates, lipids, colorants, metallic ions.
Elevated concentration of proteic material means the maintenance of the initial ratio of proteic material when a sample of the complex mixture is submitted to a treatment for the extraction of the whole content of the proteic material.
Analyte signifies the sample containing proteic material whose presence is the objective of the analysis.
Matrix includes the aromatic compounds with carboxyl groups that when strongly absorbing UV (ultraviolet) radiation (266, 337 and 335 nm) on the laser wavelength used, free protons for the ionisation of the analyte.
Low levels of impurities of the matrix-analyte combination means the substantial absence of interferents in the stage of sample preparation for spectrometric analysis.
Adequate matrix-analyte molar ratio means that the concentration of the matrix presents a molar excess in relation to the sample, saturating it and thus guaranteeing efficient ionisation of the analyte in groups according to their masses whilst they traverse the free field region (TOF analyser).
External calibration standard (Calmix 3, Applied Biosystems) represents a mixture of proteins (bovine insulin, thioredoxin and equine myoglobin) used for the verification of the masses and calibration of the instrument, in this case a mass spectrometer.

The first stage of the method of the present invention is the preparation of the sample, in other words, the extraction of the proteic content of the analyte to be analysed. Various solvents (water, ethanol, acetonitrile, trifluoroacetic acid) combined or not and conditions for the treatment of the sample (concentration, temperature and extraction time, concentration and volume of the extraction solution, number of extractions; agitation time; time, temperature and velocity of centrifugation) may be used to extract the proteic content, such as, for example, the procedure described in Example 1. This stage is of vital importance for an efficient result of the analysis by mass spectrometry. It is desirable that all interferents (carbohydrates, lipids, colorants, metallic ions) be removed and merely the proteic material (in elevated concentrations) forms the analyte. In a preferred embodiment of the present invention, two extractions were performed for the analyte to be considered adequate for the analysis. Furthermore, the experimental conditions for the treatment of the sample were carefully tested and optimised as demonstrated in Example 1.

The second stage of the method of the present invention consists in the analysis of the proteic material extracted from the sample (analyte) by MALDI-TOF mass spectrometry. This technique is based on mixing the analyte with an organic acid (matrix) that greatly absorbs UV radiation (266, 337 and 335 nm) or IR (2.94, 2.79 and 10.6 μm) on the laser wavelength employed. The matrix-analyte mixture is applied over a laser probe (metallic plaque). The solvent is evaporated at ambient temperature or by means of a flow of cold or hot air, leading to the crystallisation of the matrix and incorporation of the analyte molecules. When the laser radiation falls over a determined region of the crystal it is absorbed and the matrix and analyte desorb in gaseous phase. Abundant and intact analyte ions with a general composition [(M+H)⁺, (2M+H)⁺, (M+2H)⁺] and their analogue negative ions [(M−H)⁻, (2M-H)⁻, (M−2H)⁻] are formed during this process. Following this, these ions are accelerated by a power (V) and accelerate through an accelerator tube with a length of 1-2 metres. All the ions receive the same kinetic energy during the acceleration. However, because they possess different mass/charge rates (m/z) they separate into groups according to their velocities whilst they traverse the free field region (TOF analyser). The sample preparation (matrix-analyte mixture) is a critical stage to obtain success in the MALDI-TOF-MS analysis, because it may affect considerably the quality of the data obtained (mass spectrums). Two main parameters contribute considerably to the quality of the data: (a) high levels of impurities present in the solutions used in the preparation of the matrix and sample; and (b) matrix/sample molar rate. The matrix solutions are generally prepared in water, water-acetonitrile or water-mixtures of alcohols in a concentration of 5-10 mg/ml, depending on the solubility properties of the matrix. The analyte is prepared in a saturation concentration of around 0.1 g/l and in a solvent that is miscible to the matrix solution (TFA (trifluoroacetic acid) 0.1% is generally used for proteins). The solutions of the matrix and analyte are mixed to obtain an adequate final molar rate, as defined above at around 5000:1 and a final volume of 0.5 to 2 μl. The different types of matrix and their preparation are known and normally indicated by the manufacturer of the equipment being used. The documents U.S. Pat. No. 6,111,251, U.S. Pat. No. 6,057,543, U.S. Pat. No. 6,287,872, U.S. Pat. No. 6,278,794 and U.S. Pat. No. 6,265,715 are examples where detailed information may be found concerning the MALDI-TOF-MS spectrometry technique and the materials and conditions appropriate for each type of macromolecule to be analysed.

Whilst specialised technicians in this field may without requiring much experimentation—vary the concentrations, conditions for performing the tests and materials the parameters used for this present invention were defined in accordance with example 2. In the present invention, a solution of ferrulic acid (4-hydroxy-3-methoxycynamic acid) in a concentration of 25 mg/ml in acetone was used as a matrix and a nitrogen laser (337 nm) was employed in radiating the analyte. The mixture was of 4 μl of sample 1 (example 1), in various concentrations, with 4 μl of the matrix solution, following which 1 μl of the mixture was applied to the plaque. For each feed being studied, various dilutions of sample 1 with an aqueous solution of trifluoroacetic acid (TFA) 0.1% were made (sample 1: TFA 0.1%-2:2, 2:6, 2:8, 2:10, 2:15, 2:18, 2:20) before mixing with the matrix, with the aim of determining the optimum concentration of each sample for analysis. An external calibration standard (Cal mix 3, Applied Biosystems) was employed to verify the masses. This procedure was satisfactory for detecting the presence or absence of proteins, whether of animal or vegetable origin in the majority of the feeds, with no need for RP-HPLC and N-terminal sequencing techniques.

The examples that follow aim to illustrate the preferred embodiments of the invention. It is evident to specialists in this matter that the procedures described in the examples represent manners of executing the invention and, therefore, any modifications to the conditions, stages or materials used that maintain the essential characteristics and that remain within the functional limits of the method of detection being proposed here are part of the present invention.

### EXAMPLES

### Example 1

### Preparation of a Sample from Feed

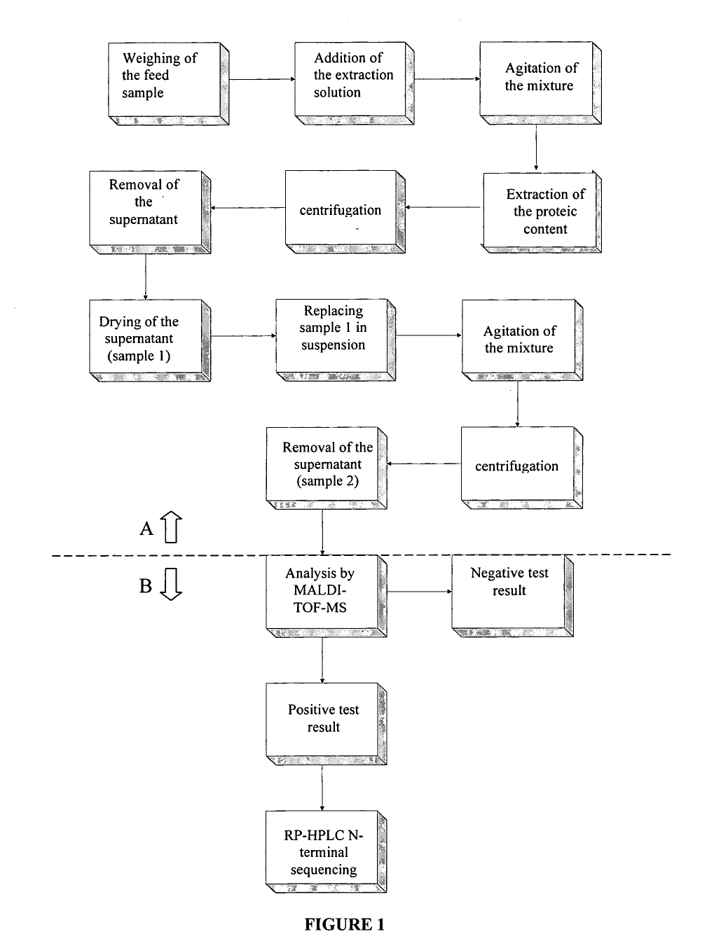

A 2 ml Eppendorf tube is used to weigh 0.3 g of feed to which is added 2.0 ml of a 1:1 mixture of an aqueous solution of trifluoroacetic acid (TFA) 0.1% and a solution of TFA 0.1% in acetonitrile. The resulting mixture is agitated for 30 seconds and kept standing at 4° C. for 24 hours to allow the extraction of the proteic material. Following this, the mixture is centrifuged at 13.200 rpm and 22° C. for 5 minutes. The liquid phase [supernantant] is then removed and dried in vacuum by lyophilisation (sample 1). The solid phase (precipitate) is discarded. After replacing sample 1 in a suspension of 1.0 ml of an aqueous solution of TFA 0.1%, the mixture is agitated for 60 seconds and centrifuged again at 13.200 rpm and 22° C. for 5 minutes. The [supernantant] (sample 2) is removed and stored for later analysis of proteic composition. The precipitate is discarded. FIG. 1, Part A, schematically shows the extraction stage of the proteic content of the sample.

Table 1 shows the set of 185 feeds analysed through the method of the present invention, accompanied by their respective codes.

TABLE 1
Samples of commercial feed analysed by
the method of the invention.

| Sample/n. | Sample/Code |
|---|---|
| 1 | A1 |
| 2 | A2 |
| 3 | A3 |
| 4 | A4 |
| 5 | A5 |
| 6 | A6 |
| 7 | A7 |
| 8 | A8 |
| 9 | A9 |
| 10 | A10 |
| 11 | B1 |
| 12 | B2 |
| 13 | B3 |
| 14 | B4 |
| 15 | B5 |
| 16 | B6 |
| 17 | B7 |
| 18 | B8 |
| 19 | C1 |
| 20 | C2 |
| 21 | C3 |
| 22 | D1 |
| 23 | D2 |
| 24 | D3 |
| 25 | D4 |
| 26 | D5 |
| 27 | D6 |
| 28 | D7 |
| 29 | D8 |
| 30 | D9 |
| 31 | D10 |
| 32 | D11 |
| 33 | D12 |
| 34 | E1 |
| 35 | E2 |
| 36 | E3 |
| 37 | E4 |
| 38 | E5 |
| 39 | E6 |
| 40 | F1 |
| 41 | F2 |
| 42 | F3 |
| 43 | F4 |
| 44 | F5 |
| 45 | F6 |
| 46 | F7 |
| 47 | F8 |
| 48 | F9 |
| 49 | F10 |
| 50 | F11 |
| 51 | F12 |
| 52 | F13 |
| 53 | F14 |
| 54 | F15 |
| 55 | F16 |
| 56 | G1 |
| 57 | G2 |
| 58 | G3 |
| 59 | G4 |
| 60 | G5 |
| 61 | H1 |
| 62 | H2 |
| 63 | H3 |
| 64 | H4 |
| 65 | H5 |
| 66 | H6 |
| 67 | H7 |
| 68 | H8 |
| 69 | H9 |
| 70 | H10 |
| 71 | H11 |
| 72 | H12 |
| 73 | H13 |
| 74 | H14 |
| 75 | H15 |
| 76 | I1 |
| 77 | I2 |
| 78 | I3 |
| 79 | I4 |
| 80 | I5 |
| 81 | I6 |
| 82 | I7 |
| 83 | I8 |
| 84 | I9 |
| 85 | I10 |
| 86 | I11 |
| 87 | I12 |
| 88 | I13 |
| 89 | I14 |
| 90 | I15 |
| 91 | I16 |
| 92 | I17 |
| 93 | I18 |
| 94 | I19 |
| 95 | I20 |
| 96 | I21 |
| 97 | I22 |
| 98 | I23 |
| 99 | I24 |
| 100 | I25 |
| 101 | I26 |
| 102 | I27 |
| 103 | I28 |
| 104 | I29 |
| 105 | I30 |
| 106 | J1 |
| 107 | J2 |
| 108 | J3 |
| 109 | J4 |
| 110 | J5 |
| 111 | K1 |
| 112 | K2 |
| 113 | K3 |
| 114 | K4 |
| 115 | K5 |
| 116 | K6 |
| 117 | K7 |
| 118 | K8 |
| 119 | K9 |
| 120 | K10 |
| 121 | K11 |
| 122 | K12 |
| 123 | K13 |
| 124 | K14 |
| 125 | K15 |
| 126 | K16 |
| 127 | K17 |
| 128 | K18 |
| 129 | K19 |
| 130 | K20 |
| 131 | K21 |
| 132 | K22 |
| 133 | K23 |
| 134 | K24 |
| 135 | K25 |
| 136 | K26 |
| 136 | L1 |
| 137 | L2 |
| 138 | L3 |
| 139 | L4 |
| 140 | L5 |
| 141 | L6 |
| 142 | L7 |
| 143 | L8 |
| 144 | L9 |
| 145 | L10 |
| 146 | L11 |
| 147 | L12 |
| 148 | L13 |
| 149 | L14 |
| 150 | M1 |
| 151 | M2 |
| 152 | M3 |
| 153 | M4 |
| 154 | M5 |
| 155 | M6 |
| 156 | M7 |
| 157 | M8 |
| 158 | M9 |
| 159 | M10 |
| 160 | N1 |
| 161 | N2 |
| 162 | N3 |
| 163 | N4 |
| 164 | N5 |
| 165 | N6 |
| 166 | N7 |
| 167 | N8 |
| 168 | N9 |
| 169 | N10 |
| 170 | N11 |
| 171 | N12 |
| 172 | N13 |
| 173 | N14 |
| 174 | N15 |
| 175 | N16 |
| 176 | N17 |
| 177 | N18 |
| 1178 | N19 |
| 179 | N20 |
| 180 | N21 |
| 181 | N22 |
| 182 | N23 |
| 183 | N24 |
| 184 | N25 |
| 185 | N26 |

### Example 2

### Analysis of the Proteic Content by the MALDI-TOF-MS Technique.

The 185 samples of the commercial feed listed in table 1 were analysed by mass spectrometry employing the MALDI-TOF technique for the detection of proteins of animal origin, specifically myoglobin and haemoglobin.

A Voyager DE-STR (Applied Biosystems, Framingham, Mass.) mass spectrometer was used. The following experimental parameters were employed for performing the analyses:
Matrix: ferrulic acid 25 mg/ml;
Mode: linear;
Acceleration voltage: 25 kV;
Laser N₂: 2470-2770 μJ cm⁻²;
Pressure at the ion source: 5.5×10⁻¹⁰ MPa (8×10⁻⁸ torr);
Pressure at the detector: 6.2×10⁻¹¹ MPa (9×10⁻⁹ torr).

FIG. 1, Part B, schematically shows the stages of analysing the proteic content of the feed in accordance with the present invention.

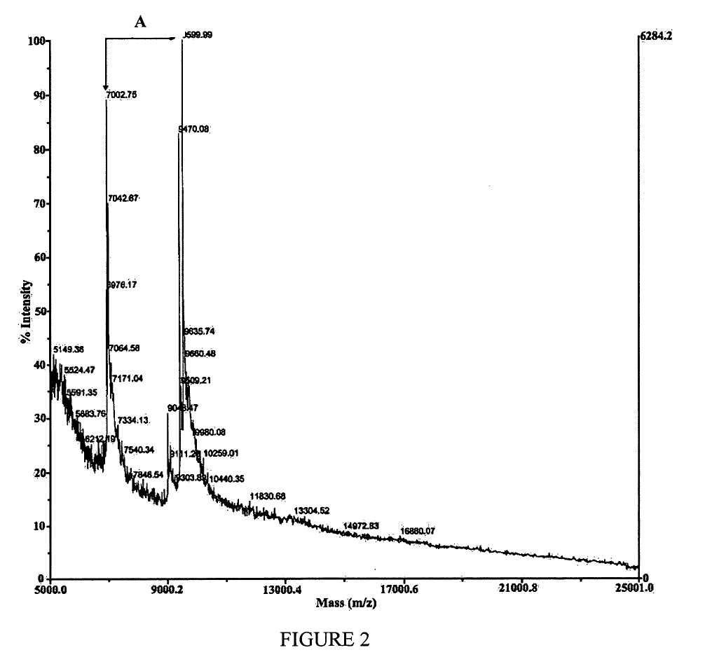

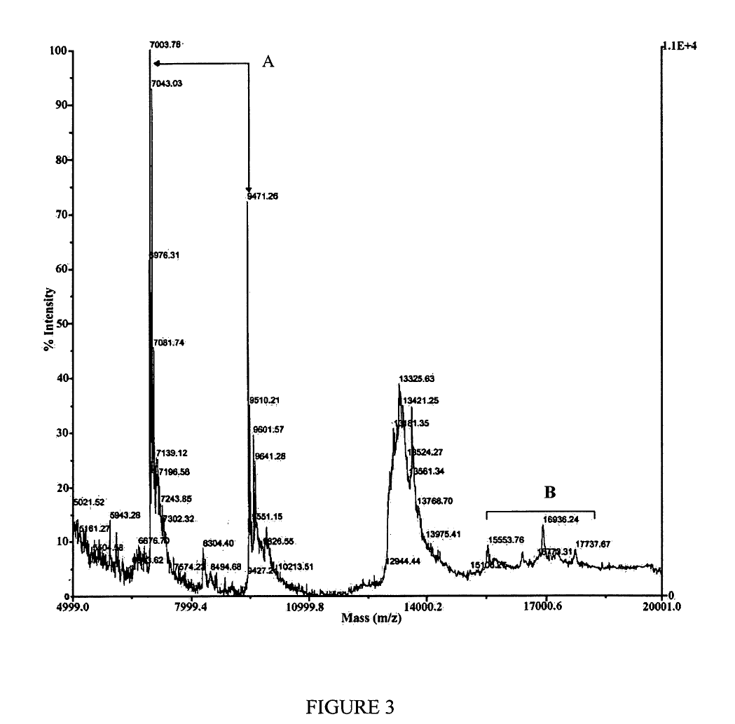

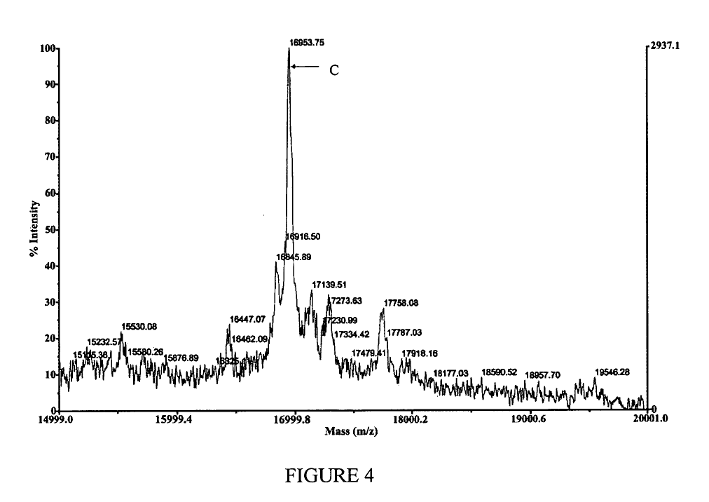

FIGS. 2 to 4 show examples of these spectrum, which were taken from two distinct feeds: A5 and G2. In the case of sample A5, the absence of peaks in the region from 15 to 17 kDa can be noted, thus indicating that this feed does not include animal protein in its composition. Furthermore, the appearance of the peaks 7,002.75 Da and 9,599.99 Da can be observed in its spectrum, which characterise the presence of protein of vegetable origin (wheat and maize, respectively) in its composition. It must be stressed that all the feeds that did not present peaks in the region from 15 to 17 kDa had practically the same sample profile as A5. In the case of sample G2, the presence of porcine myoglobin in its composition was confirmed by the appearance of peak 16,953.52 Da (average value; n. of repetitions: 6) (see FIG. 4). Three other peaks were observed in its mass spectrum (7,003.76 Da, 9,471.26 Da and 13,325.63 Da respectively). Peaks 7,003.76 Da and 9,471.26 Da attest to the presence of vegetable proteins (wheat and maize) in the composition of feed G2. The presence of peak 13,325.63 Da can be attributed to a possible degradation of part of the porcine myoglobin present in the mixture. Separation of the components by RP-HPLC allows confirmation of this supposition. Other peaks of low intensity (region from 15 to 17 kDa) were also observed in the mass spectrum of feed G2, suggesting that the sample also contained traces of gallinaceous haemoglobin and myoglobin in its composition. It is likely that, due to the concentrations of these proteins probably being below the detection limits of the equipment, it is not possible to verify their presence in a precise manner. The detection limit of this method may be estimated by the calculation of the number of mols of myoglobin of the spectrum in FIG. 4, corresponding to the detection of such protein in feed G2 by the MALDI-TOF-MS technique, as demonstrated below:

$$\begin{matrix}{m}_{feedLA} = 0 \text{,} 2600 g \\ {m}_{precipitated} = 0 \text{,} 2337 g ( after {1}^{st} extraction ) \\ {m}_{MYG} = {m}_{feedLA} - {m}_{precipitated} = 0 \text{,} 0263 g \\ [ MYG ] = 26 \text{,} 3 mg \text{/} ml ( {2}^{nd} extraction ) \\ [ MYG ] = \frac{2 μl \times ( 26 \text{,} 3 mg \text{/} ml )}{10 μl} 5 \text{,} 26 mg \text{/} ml \\ ( diluted with TFA 0 \text{,} 1 \% ) \\ [ MYG ] = \frac{4 μl \times ( 5 \text{,} 26 mg \text{/} ml )}{8 μl} = 2 \text{,} 63 mg \text{/} ml \\ = 2 \text{,} 63 μg \text{/} μl ( diluted with the matrix )\end{matrix}$$

$$1 mol MYG -- -- -- 16 \text{,} 953.52 g {n}_{MYG} = 0 \text{,} 155 nmoles \\ ⟵ \\ {n}_{MYG} 2 \text{,} 63 \times {10}^{- 6} g$$

Similar calculations were made for the other samples of feeds that presented positive test results, with results of the same magnitude being obtained.

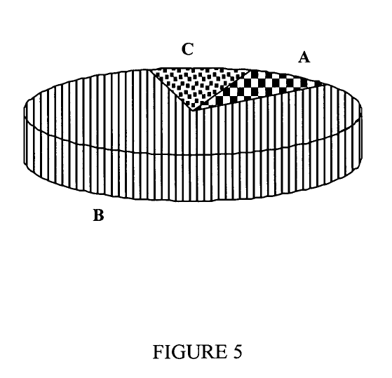

FIG. 5 shows a graphic representation of the overall result of the analyses of 185 samples of commercial feed available on the Brazilian market. The graph was generated from the data obtained from the mass spectrums of these samples. It can be noted that, of the total samples analysed, 9% of the feeds revealed the presence of animal protein in their composition (positive test results). 81% of the feeds proved to be adequate for feeding ruminants, since no animal protein was observed in these samples (negative test results). Around 10% of the samples did not provide conclusive data in their respective mass spectrums, possibly due to the presence of substances such as lipids or pigments, that interfere with the results by MALDI-TOF-MS. In this case, the separation of the interferents by methods such as RP-HPLC is recommended before submitting the sample to a further analysis by mass spectrometry.

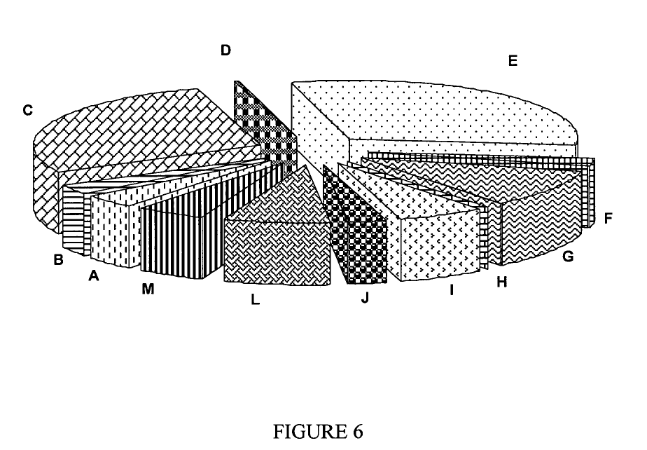

A summary of the mass spectrum obtained from all the samples of feed analysed is represented in FIG. 6, which shows the mass region of the peaks found in these spectrum: 5,000, 6,000, 8,000, 9,000, 10,000, 11,000, 13,000, 14,000, 15,000, 16,000 and 17,000 Da. It can be seen that the majority of the samples possess peaks in the region from 7,000 to 9,000 Da, which indicate the presence of vegetable proteins (wheat and maize) in the composition of such feeds. The presence of different types of myoglobin (bovine, equine, porcine and gallinaceous) in some feeds was attested by the peaks observed in the region from 16 to 17 kDa. It should be stressed that the majority of these feeds that presented peaks in the region of 16 kDa also presented a peak in the region of 13 kDa. The peaks observed in the region of 16.9 kDa indicate the presence of bovine, equine or swine myoglobin. Some feeds presented peaks in the region of 17.2 kDa which suggest the presence of gallinaceous myoglobin. Peaks in the region of 15 kDa were also observed in some feeds, pointing to the presence of haemoglobin in the composition of these feeds as well.

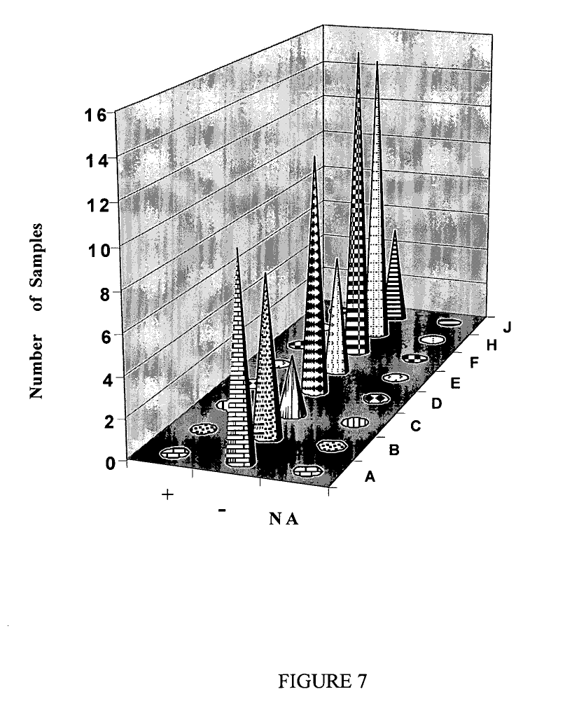

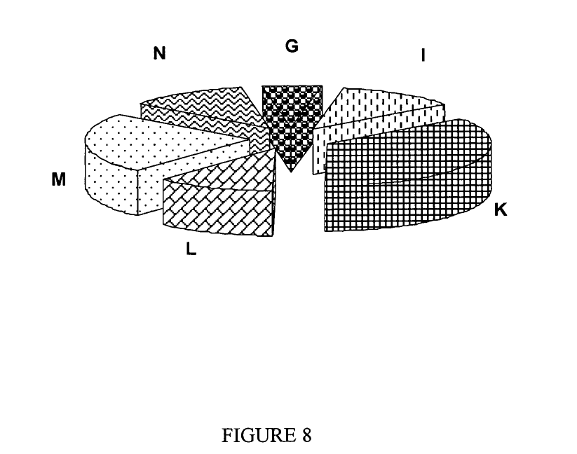

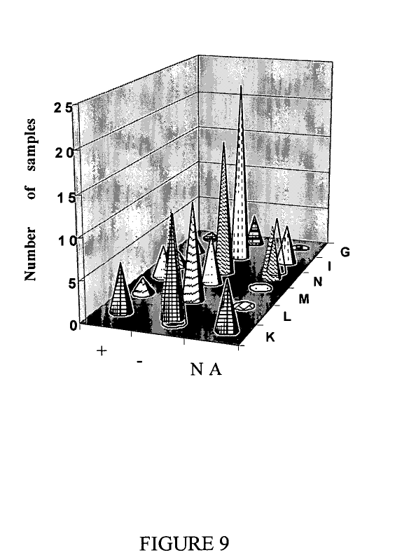

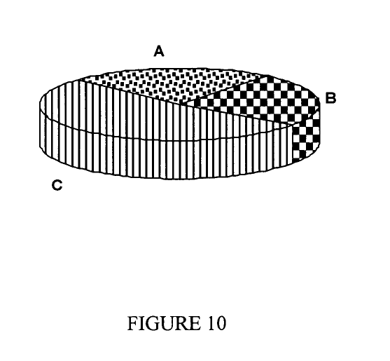

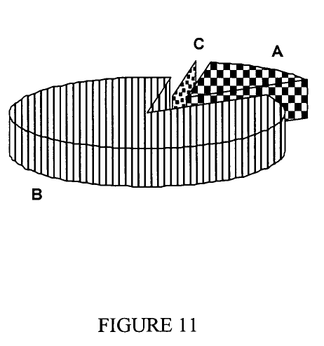

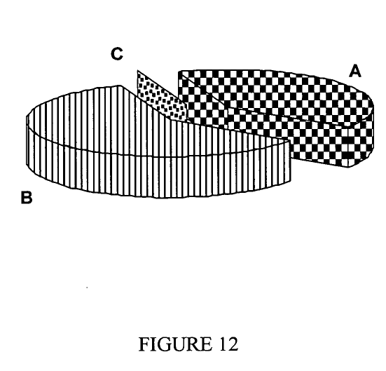

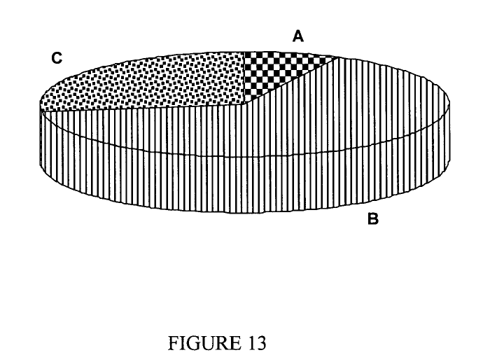

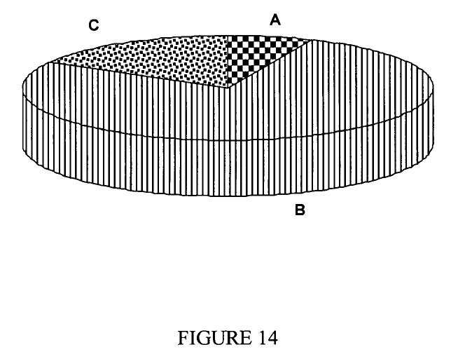

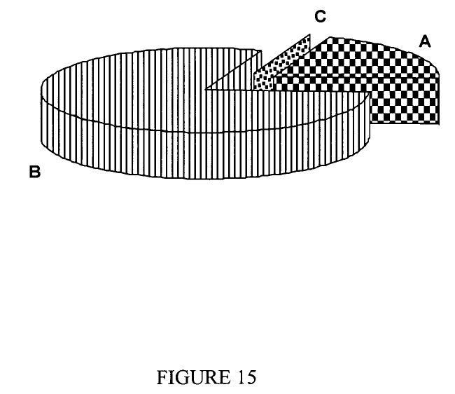

The MALDI-TOF-MS technique was used to analyse a total of fourteen groups of commercial feed. In eight of these groups (B, A, F, J, E, H, C and D), the presence of animal protein was not detected in the samples. FIG. 7 shows the total number of samples analysed for each of these eight groups and the result of the respective analyses, disposed in three categories: samples with positive test results (presence of animal protein), samples with negative test results (absence of animal protein) and samples with a non-conclusive analysis (those whose mass spectrums were not consistent, probably because of interferential substances, such as lipids and pigments). Samples with positive test results were found in six (G, I, K, L, M and N) of the fourteen groups of feed studied. The percentage of these samples in each one of the six groups is represented in FIG. 8. It can be noted that the samples of the groups K and M where those that presented the most contamination by animal protein. FIG. 9 shows the overall result of the analyses of each one of these six groups of feed, where the total number of samples analysed by group are listed and the set of results obtained by each group is disposed in three categories (positive test results, negative test results and samples with a non-conclusive analysis). These results were separated by group for better evaluation and represented in percentage in FIGS. 10 to 15.

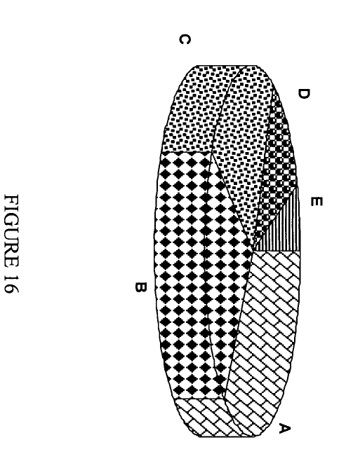

FIG. 16 shows the distribution of the feeds with positive test results by type of myoglobin. Three main types of myoglobin can be noted in the samples analysed: porcine, bovine and equine. Furthermore, 4 samples presented one of the polymorphic forms of bovine myoglobin (MAAQ→AAEK) and one sample presented a polymorphic form of equine myoglobin (D→N).

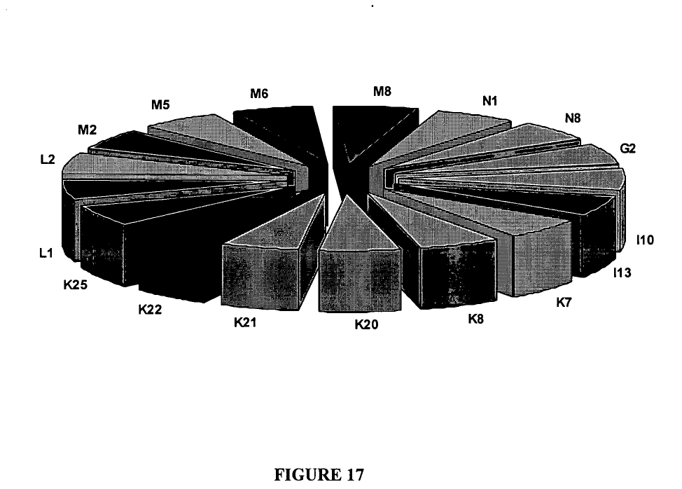

The results presented in FIGS. 16 and 17 were obtained comparing the experimental values of the masses of the peaks obtained from the mass spectrum of the feeds analysed (region of 16 kDa) as shown on Table 2 with the standard mass values of the different types of haemoglobin and myoglobin, including their polymorphic forms (see: http://www.expasy.ch), as shown on Tables 3 and 4.

TABLE 2
Masses of the peaks obtained from the mass spectrum of the
feed analysed (region of 16 kDa)

| | Average mass of | Average mass of experimental |
|---|---|---|
| Feed | experimental peak* | peak/MYG (myoglobin) |
| G2 | 16,953.52 ± 3.78 | 16953.42/MYG porcine |
| I10 | 16,967.28 ± 4.46 | 16953.42/MYG porcine |
| I13 | 16,944.02 ± 2.21 | 16944.36/MYG bovine (MAAQ---AAEK) |
| K7 | 16,948.18 ± 1.81 | 16946.4/MYG bovine |
| K8 | 16,962.27 ± 4.96 | 16953.42/MYG porcine |
| K20 | 16,956.52 ± 1.79 | 16953.42/MYG porcine |
| K21 | 16,955.33 ± 6.25 | 16953.42/MYG porcine |
| K22 | 16,950.29 ± 3.30 | 16950.49/MYG equine (D → N) |
| K25 | 16,944.53 ± 3.86 | 16944.36/MYG bovine (MAAQ → AAEK) |
| L1 | 16,944.95 ± 3.22 | 16944.36/MYG bovine (MAAQ → AAEK) |
| L2 | 16,947.14 ± 2.45 | 16946.4/MYG bovine |
| M2 | 16,943.33 ± 1.69 | 16944.36/MYG bovine (MAAQ → AAEK) |
| M5 | 16,947.27 ± 7.10 | 16946.4/MYG bovine |
| M6 | 16,951.84 ± 6.78 | 16951.48/MYG equine |
| M8 | 16,951.65 ± 4.64 | 16951.48/MYG equine |
| N1 | 16,946.16 ± 5.21 | 16946.4/MYG bovine |
| N8 | 16,956.77 ± 2.14 | 16953.42/MYG porcine |

*n = six repetitions

TABLE 3
Standard mass values of the different types of haemoglobin

| Haemoglobin | Molecular Mass/Da |
|---|---|
| Species | Molecular mass/Da |
| Ovine (chain α) | 15,033.17 |
| Polymorphic forms: | |
| G → S | 15,063.19 |
| D → Y | 15,081.26 |
| L → H | 15,057.15 |
| N → S | 15,006.14 |
| Ovine (chain β) | 16,073.44 |
| Polymorphic forms: | |
| N → S | 16,046.41 |
| N → A | 16,030.41 |
| P → A | 16,047.40 |
| MK → VQ | 16,041.33 |
| N → S | 16,046.41 |
| D → E | 16,087.47 |
| K → R | 16,101.45 |
| Porcine (chain α) | 15,039.14 |
| Porcine (chain β) | 16,034.37 |
| Polymorphic forms: | |
| N → D | 16,035.35 |
| Gallinaceous (chain α) | 15,297.68 |
| Polymorphic forms: | |
| RVD → TGG | 15,142.48 |
| A → T | 15,327.71 |
| E → K | 15,296.74 |
| V → I | 15,311.71 |
| K → N | 15,283.61 |
| Gallinaceous (chain β) | 16,334.93 |
| Equine (chain α) | 15,114.28 |
| Polymorphic forms: | |
| Y → F | 15,098.28 |
| K → Q | 15,114.24 |
| Equine (chain β) | 16,008.29 |
| Bovine (chain α) | 15,053.18 |
| Polymorphic forms: | |
| H → Y | 15,079.21 |
| N → S | 15,026.15 |
| Bovine (chain β) | 15,954.39 |
| Polymorphic forms: | |
| G → S | 15,984.42 |
| K → H | 15,963.36 |
| D → G | 15,896.35 |
| S → T | 15,968.42 |
| K → N | 15,940.32 |
| K → Q | 15,954.35 |

TABLE 4
Standard mass values of the different types of myoglobin
Myoglobin

| Species | Molecular mass/Da |
|---|---|
| Ovine | 16,923.36 |
| Polymorphic forms: | |
| E → Q | 16,922.01 |
| Porcine | 16,953.42 |
| Gallinaceous | 17,290.86 |
| Polymorphic forms: | |
| DQ → NE | 17,290.86 |
| E → D | 17,276.83 |
| N → Q | 17,304.89 |
| Equine | 16,951.48 |
| Polymorphic forms: | |
| D → N | 16,950.49 |
| Bovine | 16,946.40 |
| Polymorphic forms: | |
| L → A | 16,904.32 |
| IPV → VIP | 16,946.40 |
| DFG → NFA | 16,959.44 |
| MAAQ → AAEK | 16,944.36 |

1. Method for detecting proteins of animal origin in complex mixtures comprising the following stages:
(i) extraction of proteic material in high concentration from a sample of the initial complex mixture in a manner as to substantially remove all interferents;
(ii) preparation of the matrix-analyte in a manner as to maintain low levels of impurities and an adequate matrix-analyte molar ratio;
(iii) analysis of the material obtained in the prior stage by MALDI-TOF mass spectrometry;
(iv) optionally, fractionation of the samples or isolation of the components by RP-HPLC and identification of the components by means of automatic sequencing of the N-terminal region and sequencing of its peptidic fragments by liquid chromatography coupled to mass spectrometry (LC/MS/MS).

2. Method according to claim 1 wherein the complex mixture consists of animal feed.

3. Method according to claim 1 wherein the protein of animal origin is myoglobin and haemoglobin.

4. Method according to claim 1 wherein the extraction of the proteic material in stage (i) is processed through a solvent selected from the group consisting of water, ethanol, acetonitrile, trifluoroacetic acid or their combinations.

5. Method according to claim 4 wherein the solvent is a mixture of an aqueous solution of trifluoroacetic acid with a solution of trifluoroacetic acid in acetonitrile.

6. Method according to claim 1 wherein the matrix in stage (ii) consists of a solution in a concentration of 5-10 mg/ml, prepared from an organic acid which heavily absorbs UV or IR radiation on the laser wavelength employed, in a solvent selected from the group consisting of water, water and acetonitrile or mixtures of water and organic solvents.

7. Method according to claim 6 wherein the matrix consists of an aqueous solution of ferrulic acid.

8. Method according to claim 1 wherein the analyte of stage (ii) is prepared in a solvent which is miscible with the solution of the matrix.

9. Method according to claim 8 wherein the solvent is trifluoroacetic acid.

10. Method according to claim 1 characterised by the fact of using in stage (iii) an external calibration pattern for the verification of the masses and the detection of the presence or absence of proteins, whether of animal or vegetable origin.

11. Use of the method defined in claim 1 in the detection of proteins of animal origin in feed for ruminants characterised by the fact of allowing the interruption of the transmission of Transmittable Spongiform Encephalopathies.

12. Use according to claim 11 wherein the Transmittable Spongiform Encephalopathy is Bovine Spongiform Encephalopathy.
